(19) United States
(12) Patent Application Publication
(10) Pub. No.: US 2023/0365508 A1
(43) Pub. Date: Nov. 16, 2023

(54) Open Time Additive

(71) Applicants: Chen LIU, Visp (CH); Kathryn Neely, Visp (CH); Patrick Flaherty, Visp (CH); Kevin Janak, Visp (CH); Rochelle Bradford, Visp (CH); ARXADA AG, Visp (CH)

(72) Inventors: Chun Liu, Sugar Hill, GA (US); Kathryn Neely, Alpharetta, GA (US); Patrick Flaherty, Cumming, GA (US); Kevin Janak, Blauvelt, NY (US); Rochelle Bradford, Decatur, GA (US)

(21) Appl. No.: 18/026,236

(22) PCT Filed: Sep. 14, 2021
(86) PCT No.: PCT/US2021/050173

§ 371 Date: Mar. 14, 2023

Related U.S. Application Data
(60) Provisional application No. 63/077,818, filed on Sep. 14, 2020.
(60) Provisional application No. 63/232,851, filed on Aug. 13, 2021.

(51) Int. Cl. C07D 233/74; C09D 11/023; C09D 11/033; C09D 5/02

(52) U.S. Cl. CPC C07D 233/74; C09D 11/023; C09D 11/033; C09D 5/024

(57) ABSTRACT

The present disclosure is generally directed to an open time additive composition that includes at least two, different open time additives having the structure of Compound I, a salt thereof, or both. Compound I having the following formula:

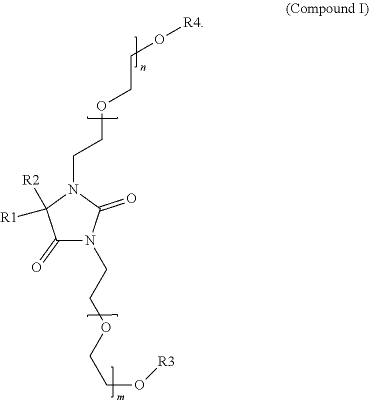

### BACKGROUND

Paints are widely used across many industries and may generally be understood to encompass a carrier that includes a pigment. However, this general view neglects that paint, especially architectural paints, also provide a protective barrier or cover to surfaces. Architectural paints may benefit from improved workability (e.g., open time) where the paint has yet to dry and may be spread further (e.g., using a brush or roller). As such, paint compositions can vary widely depending on the application.

While open time can be defined in a number of ways, the term is generally used to indicate the time a paint film allows for smooth integration of subsequently applied paint and/or the time a coating remains workably wet before setting. As already discussed, open time can be an important aspect in characterizing paints since the open time can lead to reduced overlapping of coating defects, reduced labor costs, and/or reduced material costs for fixing defects.

Some known options for adjusting open time can include increasing water content, using glycol or glycerol esters, or using additives that can increase costs significantly. Most of these solutions may only provide low/marginal open time extension and can have deleterious effects on other aspects of the paint performance. Depending on region, the known options could also be listed as a volatile organic compound (VOC), which is under much global scrutiny.

Allowing for sufficient time to paint, repair, or cover architectural coatings continues to be a challenge, especially in dry environments. Additionally, paints that have a low pigment volume concentration (PVC), such as a high solid content, and regions with strict VOC regulations can compound the challenge.

Needed still in the art are architectural paints that include an open time additive in an effective amount to maintain workability under various conditions. Additionally, due to variations in environmental conditions (e.g., humidity), paints that include an open time additive that can be modified to adjust the workability may provide additional benefits for manufacturers and consumers. For instance, customizing paint formulations by adjusting the open time additive may lead to cost savings, especially in large construction projects.

### SUMMARY

In general, the present disclosure is directed to an open time additive composition, e.g., for use with architectural paints. Architectural paints may be considered as different from other paints, dyes or compositions including pigments in that architectural paints can provide a coating for covering a surface and/or a material. Thus, generally, architectural paints can be used without the architectural paints modifying the surface and/or the material to which they are applied. In contrast, dyes or other pigment compositions may be used to incorporate the dye or a portion of the dye (e.g., the pigment/colorant) into the material. Due at least in part to these differences, architectural paints may benefit from additives that can act to increase the open time of the architectural paint. In contrast, increasing the open time of dyes or other pigment compositions could lead to undesirable bleeding. Example implementations of the present disclosure can include an open time additive composition that includes at least two, different open time additives, namely a first open time additive and a second open time additive, having the formula of Compound I:

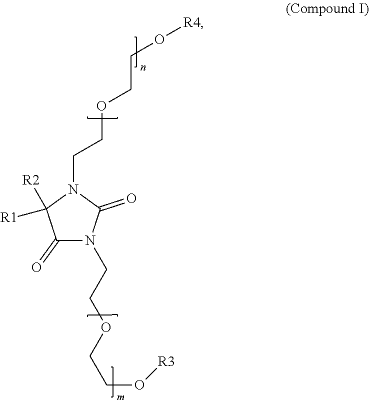

wherein, for both the first and second open time additives, m is an integer no less than zero (0), n is an integer no less than zero (0), and R1 and R2 are independently a branched or linear carbon chain having no less than one (1) and no more than forty (40) carbon atoms,

wherein, for both the first and second open time additives, each carbon atom in R1 and R2 is independently substituted with one or more hydrogen atom, one or more hydroxyl group, one or more other carbon atom in the branched or linear carbon chain, an aryl group, or a combination thereof,

wherein, for both the first and second open time additives, each of R3 and R4 are independently a hydrogen atom or an ester group, such as an oleate group,

wherein, for the first open time additive, a sum of m and n is no greater than five, and

wherein, for the second open time additive, the sum of m and n is no less than fifteen.

In a first example aspect, m may be an integer no less than five and no greater than one hundred for the second open time additive.

In a second example aspect, n may be zero for the first open time additive.

In a third example aspect, for the first open time additive, n may be no greater than five, and m is no greater than five.

In a fourth example aspect, R3, R4, or both may be the ester group for one or both of the first and second open time additives.

In a fifth example aspect, R1 and R2 may both be methyl groups for one or both of the first and second open time additives.

In a sixth example aspect, the ester group may be the oleate group having the formula of:

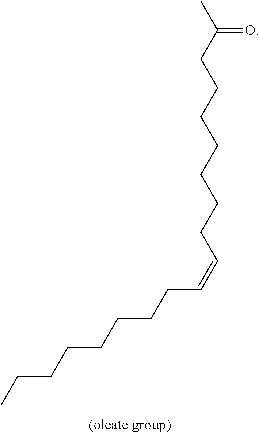 [SMILES: CCCCCCCC/C=C\CCCCCCCC(C)=O]

In a seventh example aspect, a weight ratio of the first open time additive to the second open time additive may be from 1:9 to 9:1.

In an eighth example aspect, a weight ratio of the first open time additive to the second open time additive may be from 1:3 to 3:1.

In a nineth example aspect, a weight ratio of the first open time additive to the second open time additive may be from 1:2 to 2:1.

In a tenth example aspect, the open time additive concentrate may be incorporated into an architectural paint composition that includes a solvent and a latex binder.

In an eleventh example aspect, the latex binder may include an acrylate.

In a twelfth example aspect, the solvent may be water.

In a thirteenth example aspect, a concentration of the open time additive in the architectural paint composition may be no less than about a tenth of a percent and no greater than about five percent based on the total weight of the architectural paint composition.

In a fourteenth example aspect, the architectural paint may have a solids content no less than about ten percent and no greater than about seventy percent based on the total weight of the architectural paint composition.

In a fifteenth example aspect, the open time additive and the latex binder may have a weight ratio of 1:999 to about 100:900 based on the total weight of the open time additive to the total weight of the latex binder.

In a sixteenth example aspect, the architectural paint may display an increased open time of no less than ten percent compared to a baseline open time displayed for a baseline architectural paint. The baseline architectural paint may not include the open time additive and has a relative composition for other components included in the architectural paint that is approximately the same, and the open time may be determined according to OTA test ASTM D7488-11 “Standard Test Method for Open Time of Latex Paints”.

In a seventeenth example aspect, the architectural paint may have a volatile organic compound, VOC, content of less than one thousandth of a percent based on the total weight of the architectural paint, and the VOC content may be determined according to EPA Method 24.

Each of the example aspects recited above may be combined with one or more of the other example aspects recited above in certain embodiments. For instance, all of the eighteen example aspects recited above may be combined with one another in some embodiments. As another example, any combination of two, three, four, five, or more of the eighteen example aspects recited above may be combined in other embodiments. Thus, the example aspects recited above may be utilized in combination with one another in some example embodiments. Alternatively, the example aspects recited above may be individually implemented in other example embodiments. Accordingly, it will be understood that various example embodiments may be realized utilizing the example aspects recited above.

Other features and aspects of the present disclosure are discussed in greater detail below.

### DETAILED DESCRIPTION

It is to be understood by one of ordinary skill in the art that the present disclosure is a description of exemplary embodiments only and is not intended as limiting the broader aspects of the present disclosure.

The present disclosure is generally directed to an open time additive having the structure of Compound I, a salt thereof, or both. Compound I has the following formula:

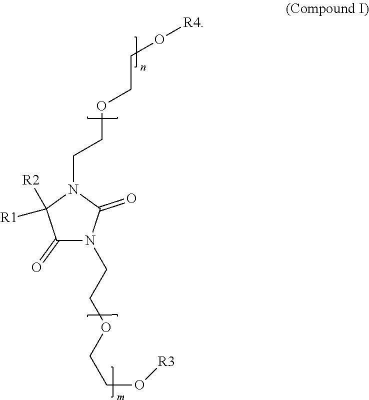

An open time additive composition may include at least two, different open time additives, namely a first open time additive and a second open time additive, having the formula of Compound I. For both the first and second open time additives: m is an integer no less than zero (0), n is an integer no less than zero (0), and R1 and R2 are independently a branched or linear carbon chain having no less than one (1) and no more than forty (40) carbon atoms; each carbon atom in R1 and R2 is independently substituted with one or more hydrogen atom, one or more hydroxyl group, one or more other carbon atom in the branched or linear carbon chain, an aryl group, or a combination thereof; and each of R3 and R4 are independently a hydrogen atom or an ester group, such as an oleate group. For the first open time additive, a sum of m and n is no greater than five (5). For the second open time additive, the sum of m and n is no less than fifteen (15).

The ester group may be an oleate group having the following formula:

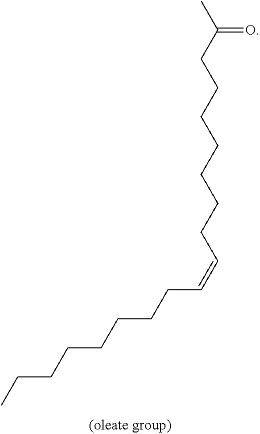 [SMILES: CCCCCCCC/C=C\CCCCCCCC(C)=O]

In certain example embodiments, the ester group may be a saturated or unsaturated C6 to C22 ester group, such as stearate (C18, saturated), oleate (C18, unsaturated), linoleate (C18, unsaturated), palmitate (C16, saturated), laurate (C12 saturated), decanoate (C10, saturated), or octanoate (C8, saturated).

Aspects of some implementations of the present disclosure can include a weight ratio of the first open time additive to the second open time additive. For example, the weight ratio of the first open time additive to the second open time additive may be from 1:9 to 9:1, such as from 1:3 to 3:1, such as from 1:2 to 2:1. Such weight ratios may advantageously increase open time when the open time additive composition is added to an architectural paint, e.g., a twenty percent (20%) increase in open time relative to an untreated control, without negatively impacting scrub resistance and with suitable stain resistance relative to a single open time additive.

The open time additive composition may be incorporated into an architectural paint. In one example implementation, the architectural paint includes a solvent, a latex binder (e.g., a polymer including one or more acrylate, vinyl acetate, vinyl chloride, and/or styrene butadiene monomers), and the open time additive. Optionally, the architectural paint can further include a dispersant and/or surfactant to improve distribution of the latex binder throughout the architectural paint. In this manner, the dispersant and/or surfactant can be used to produce a more homogenous mixture that can provide a more even coating of the architectural paint. Optionally, the architectural paint can include a thickening agent to adjust the viscosity of the architectural paint to improve adhesion of the wet paint to an applicator (e.g., a brush or roller). Optionally, the architectural paint can include one or more pigments (e.g., TiO₂) for providing a color to the architectural paint. Optionally, the architectural paint can include a cosolvent (e.g., ethylene glycol) that can improve solubility of components of the architectural paint.

An example aspect of implementations according to the present disclosure can include a low volatile organic compounds (VOCs) content. High VOCs are recognized as environmental hazards as well as demonstrating personal hazards to painters who work in confined and/or unventilated spaces. In these spaces, VOCs can collect in the air which may cause breathing issues for painters and possible health concerns. Many known paint additives that are used to modify the paint open time are known high VOCs which has posed challenges. Poor open time performance can require increased working time to correct mistakes, such as streaking that are inherent to the paint composition. Thus, improving open time while also mitigating VOC content can provide a great advantage in the cost and efficiency of paint projects as well as the health of painters.

Another aspect of example implementations can include a type of latex binder. The latex binder can include various polymers suitable for architectural paints such as an acrylates (e.g., polymethylmethacrylate), that can be formed as a homopolymer or co-polymer. For example, a co-polymer can include incorporation of another monomer (e.g., butadiene styrene). In some implementations, the acrylate can be modified to include one or more nitrile groups. Thus, latex binders can include various acrylates, acrylate butadiene styrene copolymers, and acrylonitrile butadiene styrene copolymers. Additionally, these latex binders are provided for example purposes, and additional latex binders may be used alone or in combination with implementations of the disclosure.

As an example for illustration, an implementation of the present disclosure can include an architectural paint including a latex binder with an acrylate. The acrylate can include a polymer or copolymer that includes one or more acrylate monomers. Example aspects of the acrylate polymer or copolymer can include a mass fraction of an acrylate monomer. For instance, the acrylate can include a copolymer that includes an acrylate monomer (e.g., methyl methacrylate) and a second monomer (e.g., butadiene styrene). The mass fraction of the acrylate monomer to the total weight of the copolymer can define the mass fraction. In some acrylates the mass fraction of acrylate monomer to the total weight of the copolymer can be no less than about twenty (20) wt % and no greater than about one hundred (100) wt % such as no less than about thirty (30) wt % and no greater than about eighty (80) wt %, no less than about forty (40) wt % and no greater than about seventy (70) wt %, or no less than about forty five (45) wt % and no greater than about sixty (60) wt % (e.g., one hundred (100) wt %, ninety five (95) wt %, ninety (90) wt %, eighty five (85) wt %, eighty (80) wt %, seventy five (75) wt %, seventy (70) wt %, sixty five (65) wt %, sixty (60) wt %, fifty five (55) wt %, or fifty (50) wt %). In particular, certain implementations can include an acrylate having a mass fraction of acrylate monomer to the total weight of acrylate greater than fifty (50) wt %.

For certain example implementations of the present disclosure, the open time additive can include two or more of 1,3-Bis(2-(hydroxypolyethoxy)ethyl)-5,5-dimethylimidazolidine-2,4-dione, 1,3-Bis(2-(hydroxypolyethoxy)ethyl)-5,5-dimethylimidzaolidine-2,4-dione monoester, and 1,3-Bis(2-(hydroxypolyethoxy)ethyl)-5,5-dimethylimidzaolidine-2,4-dione diester. Each of these compounds can be derived from the formula of Compound I, respectively, as follows: n is no less than one (1) and no greater than one hundred (100) and m is no less than one (1) and no greater than one hundred (100), R1 and R2 are each linear carbon chains including one (1) carbon atom substituted with three (3) hydrogen atoms (e.g., a methyl group); R1 and R2 are each linear carbon chains including one (1) carbon atom substituted with three (3) hydrogen atoms (e.g., a methyl group) and R4 or R3 is an ester group; and R1 and R2 are each linear carbon chains including one (1) carbon atom substituted with three (3) hydrogen atoms (e.g., a methyl group) and both R4 and R3 are an ester group.

In implementations where the open time additive includes the oleate group, it should be understood that the oleate group is bound such that the carbonyl carbon is linked to the terminal oxygen (at R3 and/or R4) to form an ester. Thus, the oleate group is illustrated to display the fatty acid carbon chain (seventeen (17) carbon, mono-unsaturated group) bound to the carbonyl carbon and a second bond to indicate the attachment position of Compound Ito the oleate group.

Further, in implementations of the present disclosure, it should be understood that for open time additives based on Compound I, the polymerization degrees n and m can be different (e.g., n and m may have different values such as n is four and m is five) or the same (e.g., n is four and m is four). Additionally, certain implementations may include combinations of open time additives based on Compound I such as an architectural paint including both 1,3-Bis(2-(hydroxypolyethoxy)ethyl)-5,5-dimethylimidazolidine-2,4-dione and 1,3-Bis(2-(hydroxypolyethoxy)ethyl)-5,5-dimethylimidzaolidine-2,4-dione monooleate.

Example implementations formulated according to the present disclosure may provide additional benefits for formulating low VOC architectural paints. In particular, example implementations may include a solvent that can be considered low or no VOC. For instance, water is not an organic compound and so is preferably incorporated in architectural paints of the present disclosure. In addition to water, a co-solvent can be included to improve solubility of components of the architectural paint (e.g., the open time additive, surfactants, pigments, etc.). Example co-solvents may be VOC exempt (e.g., acetone, dimethyl carbonate, methyl acetate, parachlorobenzotrifluoride, tert-butyl acetate, and propylene carbonate) or be included in lower concentrations (e.g., lower weight percentages) to limit the VOC concentration of the architectural paint.

For instance, certain implementations of the present disclosure can include architectural paints having a VOC content of less than one thousandth of a percent (<0.001%) based on the total weight of the architectural paint. VOC content can be determined using various methods, preferably example implementations can include specific VOC content determined according to EPA Method 24 for surface coatings.

Alternative methods for determining VOC content may also be used to determine VOC content in some example implementations. For instance, ASTM D6886-14 does not specifically define what constitutes a VOC ingredient based on chemical properties, but rather, implies that any components that produce a peak in a gas chromatogram are considered VOC (exempt or non-exempt). Additionally, IOS 11890-2 can be used to determine VOC content based on a pre-defined boiling point limit. As an example, if the term “VOC” is being used for compounds whose boiling points are below the boiling point limit, a marker compound of known purity and with a boiling point (BP) within ±3° C. of the defined maximum is used. So, if the EU definition for VOC is being employed (i.e. any compound with a boiling point below 250° C. is classified as VOC), tetradecane (with a BP of 252.6° C.) or a similar boiling point non-polar compound can be used as a marker compound for non-polar systems, while diethyl adipate (with a BP of 251° C.) can be used for polar systems,

Example implementations in accordance with the present disclosure may include a VOC content, as determined using one of the methods disclosed herein (e.g., EPA Method 24), of no less than one hundred thousandth of a percent (0.00001%) and no greater than one thousandths of a percent (0.001%), such as a VOC content no less than five hundred thousandths of a percent (0.00005%) and no greater than eight ten thousandths of a percent (0.0008%), or no less than on ten thousandth of a percent (0.0001%) and no greater than five ten thousandths of a percent (0.0005%). In some implementations, the VOC content can be substantially zero, for example including a substantially undetectable amount of VOC based on the analytical tool used to determine VOC content (e.g., a gas chromatograph).

For example implementations, the open time additive can be included in the architectural paint in an effective amount to produce reduced streaking, even in environments that have low humidity. For instance, the open time additive can be included at a concentration of no less than about a tenth of a percent (0.1%) and no greater than about five percent (5%) based the weight of the open time additive to the total weight of the architectural paint, such as no less than about one half of a percent (0.5%) and no greater than about four and one half percent (4.5%), no less than about one percent (1.0%) and no greater than about four percent (4.0%), no less than about one and two tenths of a percent (1.2%) and no greater than about three and one half percent (3.5%), and no less than about two percent (2%) and no greater than about three percent (3%).

Example aspects of the open time additive can include substructures of Compound I. Some example substructures can include compounds where m is no less than five (5) and no greater than one hundred (100). Another example substructure can include compounds where n is zero (0). Additional example substructures can include compounds where n is no greater than ten (10) and m is no greater than ten (10). Additionally or alternatively, further example substructures can include compounds where R3 and/or R4 are an oleate group.

Another aspect of some implementations according to the present disclosure can include a solids content no less than five percent (5%) and no greater than seventy percent (70%), such as no less than eight percent (8%) and no greater than fifty percent (50%) or no less than ten percent (10%) and no greater than thirty percent (30%) [e.g., twelve percent (12%), fourteen percent (14%), fifteen percent (15%), sixteen percent (16%), or eighteen percent (18%)] based on the total weight of the open time additive to the total weight of the latex binder.

Aspects of some implementations of the present disclosure can include a weight ratio of the open time additive to the latex binder. Advantageously, the weight ratio of the open time additive to the latex binder is no greater than 1:999 and no less than 1:9 such as no greater than 1:900 and no less than 1:9, no greater than 1:800 and no less than 1:9, no greater than 1:800 and no less than 1:90, or no greater than 1:800 and no less than 1:200 (e.g., 1:900, 1:800: 1:700 1:600, 1:500, 1:400, 1:300, 1:200, or 1:100).

As used herein, the weight ratio of the open time additive to the latex binder should be understood on the basis of the open time additive. As such, no greater than 1:999 should be read as for every one (1) weight unit of the open time additive, there is no greater than nine-hundred and ninety-nine (999) weight units of the latex binder. As another example for illustration, no less than 1:9 should be read as for every one (1) weight unit of the open time additive, there is no less than nine (9) weight units of the latex binder.

In implementations of the present disclosure, the architectural paint can include or may be formulated to include an amount of pigment. For instance, certain example architectural paints can include a pigment, the pigment including titanium dioxide (TiO₂) at a concentration of no less than fifteen (15) wt % TiO₂ and no greater than sixty (60) wt % TiO₂ based on the total weight of the architectural paint. TiO2 can be used to impart whiteness and/or opacity to example implementations and may also be included to build viscosity. In general, example implementations can include no less than fifteen (15) wt % and no greater than sixty (60) wt % TiO₂ such as no less than eighteen (18) wt % and no greater than fifty five (55) wt % TiO₂, no less than twenty (20) wt % and no greater than fifty (50) wt % TiO₂, or no less than twenty five (25) wt % and no greater than forty five (45) wt % TiO₂.

One example aspect of certain implementations can include an increase in open time resulting from the addition of the open time additive to the paint composition. To determine the increase in open time, a base paint having a composition that does not include the open time additive can be modified to produce the architectural paint, by adding an effective amount of the open time additive to the base paint. For some implementations, the addition of the effective amount of the open time additive to the base paint can produce an increase in the open time determined for the architectural paint, relative to the base paint alone, of no less than ten percent (10%), such as no less than twenty percent (20%). Open time can be determined using a variety of methods, preferably implementations according to the present disclosure can determine open time according to OTA test ASTM D7488-11 “Standard Test Method for Open Time of Latex Paints”.

Alternatively or additionally, another example aspect of certain implementations can include an increase in scrub resistance resulting from the addition of the open time additive to the paint composition. To determine the increase in scrub resistance, a testing method such as ASTM D 2486 can be used to compare number of scrubs to failure and/or exposure of a substrate material after a number of scrubs. For instance, a first coating can be applied to the substrate material using a base paint and a second coating applied to the substrate material using an architectural paint, the architectural paint having been formulated by adding an effective amount of the open time additive to the base paint. After applying an abrasive force (e.g., a scrub) to the coatings, the scrub resistance can be determined based at least in part on removal of the coating and/or exposure of the substrate material. In some implementations, addition of an effective amount of the open time additive can produce an increase in scrub resistance (relative to the base paint) of no less than a quarter of a percent (0.25%) and no greater than sixty percent (60%), such as no less than ten percent (10%) and no greater than fifty percent (50%), no less than twelve percent (12%) and no greater than forty (40%), or no less than fifteen (15%) and no greater than thirty (30%).

Implementations of the present disclosure can also include methods for adjusting the open time of a base paint (e.g., an aqueous latex paint). The method may include forming an aqueous latex paint (e.g., a water-based acrylate) with an open time additive having the structure of Compound I or a substructure of Compound I as described herein.

One example aspect forming the aqueous latex paint with the open time additive can include homogenizing the aqueous latex paint while adding the open time additive. Homogenizing can include various forms of mixing to facilitate incorporation of the open time additive with the aqueous latex paint. For instance, homogenizing can include mixing at a specified rotation per minute (RPM) the aqueous latex paint, sonicating the aqueous latex paint at a specified frequency, and/or vortexing the aqueous latex paint. In this manner, the open time additive can be incorporated throughout the aqueous latex paint to produce an architectural paint according to example implementations of the present disclosure. Thus, example implementations can further include methods for producing architectural paints, such as example architectural paints of the disclosure using example methods of the disclosure.

Another aspect of methods for producing an architectural paint can include determining a solids content for the base paint (e.g., the aqueous latex paint), and, based at least in part on the solids content, adding an amount of Compound Ito the base paint. In particular, the solids content can determine a basis for including an effective amount of the open time additive. For instance, the amount of latex binder can be determined based on the solids content and an effective amount of the open time additive can be determined according to the ratio of the open time additive to the latex binder disclosed in example implementations herein.

Certain methods for producing an architectural paint according to the present disclosure can further include modifying Compound I by adjusting the extent of polymerization (e.g., by selecting m and/or n) to modify the open time of the aqueous latex paint.

The preceding description is exemplary in nature and is not intended to limit the scope, applicability or configuration of the disclosure in any way. Various changes to the described embodiments may be made in the function and arrangement of the elements described herein without departing from the scope of the disclosure.

As used in this application and in the claims, the singular forms “a,” “an,” and “the” include the plural forms unless the context clearly dictates otherwise. Additionally, the term “includes” means “comprises.” The methods and compositions of the present disclosure, including components thereof, can comprise, consist of, or consist essentially of the essential elements and limitations of the embodiments described herein, as well as any additional or optional ingredients, components or limitations described herein or otherwise useful in nutritional compositions.

Unless otherwise indicated, all numbers expressing quantities of ingredients, properties such as molecular weight, percentages, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is recited.

As used herein, “optional” or “optionally” means that the subsequently described material, event or circumstance may or may not be present or occur, and that the description includes instances where the material, event or circumstance is present or occurs and instances in which it does not. As used herein, “wt %” and “w/w %” means by weight as a percentage of the total weight or relative to another component in the composition.

The term “about” is intended to mean approximately, in the region of, roughly, or around. When the term “about” is used in conjunction with a numerical range, it modifies that range by extending the boundaries above and below the numerical values set forth. Unless otherwise indicated, it should be understood that the numerical parameters set forth in the following specification and attached claims are approximations. At the very least, and not as an attempt to limit the application of the doctrine of equivalents to the scope of the claims, numerical parameters should be read in light of the number of reported significant digits and the application of ordinary rounding techniques.

The phrase “effective amount” means an amount of a compound that promotes, improves, stimulates, or encourages a response to the particular condition or disorder or the particular symptom of the condition or disorder.

The present disclosure may be better understood with reference to the following example.

### EXAMPLE

Various formulations were made in accordance with the present disclosure and tested for wet edge to open time ratio (WE/OT); open time (OT), and dry to touch (DTT). A standard paint with a forty percent (40%) pigment volume concentration (PVC) solids was used for the comparative test. To the blank paint, a two percent (2%) amount of an open time additive according to the present disclosure (DS 7034, DS 7036, and combinations thereof) was incorporated. Each of these formulations was characterized using OTA test ASTM D7488-11 “Standard Test Method for Open Time of Latex Paints” and the collected results are shown in Table 1. DS 7034 was an open time additive according to the present disclosure with a long chain ester, namely an oleate group, and DS 7036 was an open time additive according to the present disclosure with an intermediate chain ester, namely shorter than the oleate group of DS 7034.

TABLE 1
Open time measurements of paints treated with open time additives
with a three mil (3 mil) wet film thickness at twenty degrees Celsius
(20° C.) and thirty-five percent (35%) relative humidity.

| | Wet Edge/ | OT % | OT % |
|---|---|---|---|
| | X-Mark, | Increase | Increase |
| Sample ID | min. | Wet Edge | X-Mark |
| Blank 40 PVC Int Paint | 2/3 | | |
| Blank paint + 2% DS 7034 | 3/≤4 | 50% | 17% |
| Blank paint + 2% DS 7034/7036 (2:1) | 4/4 | 100% | 33% |
| Blank paint + 2% DS 7034/7036 (1:1) | 4/3 | 100% | 0% |
| Blank paint + 2% DS 7034/7036 (1:2) | 4/4 | 100% | 33% |
| Blank paint + 2% DS 7034 | 3/3 | 50% | 0% |

TABLE 2
Open time measurements of paints treated with open time additives
with a ten mil (10 mil) wet film thickness at twenty degrees Celsius
(20° C.) and thirty-five percent (35%) relative humidity.

| | Wet Edge/ | OT % | OT % |
|---|---|---|---|
| | X-Mark, | Increase | Increase |
| Sample ID | min. | Wet Edge | X-Mark |
| Blank 40 PVC Int Paint | 2/3 | | |
| Blank paint + 2% DS 7034 | 3/4 | 50% | 33% |
| Blank paint + 2% DS 7034/7036 (2:1) | 4/4 | 150% | 33% |
| Blank paint + 2% DS 7034/7036 (1:1) | 5/4 | 150% | 33% |
| Blank paint + 2% DS 7034/7036 (1:2) | 5/4 | 150% | 33% |
| Blank paint + 2% DS 7034 | 3/3 | 50% | 0% |

During testing, wet edge lines can be visually identified near the edge of the paint surface, and values determined based at least in part on reapplication time subtracted from the time the edge of paint can no longer be worked into body of paint. Additionally, open time can be visually identified by streaking, and values determined based on repair time subtracted from the time an “X” is visible after a paint cycle. As shown in Tables 1 and 2, all of the open time additives according to example aspects of the present disclosure extended the open time compared to the standard paint by at least one of the evaluated standards.

TABLE 3
Properties of open time additives used for comparative studies.

| | | |
|---|---|---|
| Material | DS7034 | DS 7036 |

| % Assay | | | 100 | 75 |
|---|---|---|---|---|
| Thermal Profile | | | BP > 400 C. | Decomp. 213 C. |
| | VOC Implication | US | No VOC | Low/No VOC |
| | | EU | No VOC | Low/No VOC |

To determine VOC content, for example as provided in Table 3, example methods ISO 11890-2, ASTM D6886-14 and EPA Method 24 were performed to compare the results of each method on example open time additives (OTA) DS 7034 and DS 7036. The results are collected in Table 4, which also includes thermal properties such as melting point and boiling point. The boiling point was determined based on the onset temperatures of the large endothermic event observed using dynamic scanning calorimetry (DSC) analysis and the significant weight loss that occurred in the thermogravimetric analysis (TGA) scans. For EPA Method 24, criteria for VOC was based on weight loss (corrected for water content) after 1 hour in a 110° C. oven.

TABLE 4
VOC content determined using different methods for example open time additives.

| | | | | Contributes to | |
|---|---|---|---|---|---|
| | | | Contributes | VOC per | |
| | | | to VOC per | ASTM D6886-14 | VOC |
| | Thermal Analysis Results | | ISO 11890-2 | VOC indicated | Content |
| Example | Melting Point | Boiling Point | VOC has | by peak on | EPA |
| OTA | (MP), ° C. | (BP), ° C. | BP ≥ 250° C. | Chromatogram | Method 24 |
| DS 7034 | <25 | ≥400 | No | No | No VOC |
| DS 7036 | 71 | ~213 | No* | Yes | Low/NO VOC |

*DS 7036 decomposes at 213° C., which is considered “No” VOC herein.

Example open time additives were also tested to determine the impact of the open time additives on scrub resistance. To determine scrub resistance, a standard method, ASTM D 2486, was used to determine the increase in scrub resistance with respect to a blank paint (i.e., a paint not including the open time additive). Unexpectedly, the open time additives were found to produce an increase or no decrease in scrub resistance (e.g., removal of the architectural paint from a surface). To understand the impact of the open time additives, a commercial paint was tested. A blank sample including only the commercial paint and test samples including two percent (2%) of various open time additives were compared using a freshly formulated paint. Example results are presented in Table 5, which demonstrates increases in scrub resistant with respect to the blank paint.

TABLE 5
Scrub resistance data for commercial paints
including 2% of various open time additives.

| | Scrub Resistance % |
|---|---|
| Blank Paint | 100% |
| Blank paint + 2% DS 7034 | 136% |
| Blank paint + 2% DS 7034/7036 (2:1) | 98% |
| Blank paint + 2% DS 7034/7036 (1:1) | 105% |
| Blank paint + 2% DS 7034/7036 (1:2) | 100% |
| Blank paint + 2% DS 7034 | 99% |

During testing, a coating of blank paint or the paint including two percent (2%) of the open time additives was applied to a dark substrate. After applying a similar abrasive process to each coating, loss of paint was determined based on the appearance of the substrate.

Example open time additives were also tested to determine the impact of the open time additives on stain resistance. To determine stain resistance, a standard method, ASTM D 4828, was used to determine the increase in stain resistance. To understand the impact of the open time additives, a commercial paint with various open time additives was tested. Example results are presented in Table 6, which demonstrates changes in stain resistance.

TABLE 5
Stain resistance data for commercial paints
including 2% of various open time additives.

| | | | 2% DS | | 2% DS | | 2% DS | | | |
|---|---|---|---|---|---|---|---|---|---|---|
| | 2% DS | | 7034/7036 | | 7034/7036 | | 7034/7036 | | 2% DS | |
| | 7034 | | (2:1) | | (1:1) | | (1:2) | | 7036 | |
| Pencil | 10 | (sl+) | 10 | (=) | 10 | (=) | 10 | (=) | 10 | (−) |
| Pen | 3 | (−) | 3 | (=) | 3 | (=) | 3 | (=) | 3 | (=) |
| Blue | 7 | (sl+) | 5 | (=) | 5 | (=) | 5 | (sl+) | 5 | (=) |
| Highlighter | | | | | | | | | | |
| Purple | 5 | (sl−) | 5 | (sl−) | 5 | (=) | 5 | (=) | 7 | (=) |
| Crayon | | | | | | | | | | |
| 305 Lipstick | 7 | (=) | 7 | (sl−) | 7 | (sl−) | 7 | (=) | 10 | (=) |
| 740 Lipstick | 10 | (=) | 10 | (=) | 10 | (=) | 10 | (=) | 10 | (=) |
| Mustard | 5 | (=) | 5 | (sl−) | 5 | (sl−) | 5 | (sl−) | 5 | (=) |
| Ketchup | 10 | (=) | 10 | (=) | 10 | | 10 | (=) | 10 | (=) |
| Grape Juice | 7 | (−) | 7 | (sl−) | 10 | (sl−) | 10 | (=) | 7 | (=) |
| Gravy | 10 | (=) | 10 | (=) | 10 | (=) | 10 | (=) | 10 | (=) |
| Coffee | 5 | (−) | 3 | (−) | 5 | (−) | 7 | (=) | 5 | (sl−) |

During testing, a coating of the paint including two percent (2%) of the open time additives was applied to a substrate, and a streak of each stain or soilant was applied to the coatings. After mechanically scrubbing each coating, the condition of each stain/soilant was determined based on the appearance of the substrate. The following ratings were used: “0” corresponds to no change in the original intensity of the stain or soilant; “3” corresponds to a slight change in the original intensity of the stain or soilant such that the stain or soilant is readily visible; “5” corresponds to a moderate change in the original intensity of the stain or soilant such that the stain or soilant is slightly visible; “7” corresponds to a large change in the original intensity of the stain or soilant such that the stain or soilant is barely visible; and “10” corresponds to all of the stain or soilant being removed. The comparisons in parenthesis indicate the relative difference in stain resistance at each rating between the coatings containing 2% open time additive and a blank coating with no open time additive, with an “=” corresponding the same stain resistance, an “−” corresponding a lesser stain resistance, a “sl−” corresponding a slightly lesser stain resistance, a “sl+” corresponding a slightly better stain resistance, and a “+” corresponding a better stain resistance. As may be seen in Table 6, DS 7034 had a negative impact on pen, purple crayon, grape juice, and coffee during stain testing, DS 7036 had a slightly negative impact on coffee only, and the DS 7034/DS7036 (1:2) was able to prevent the staining effects of DS7034.

These and other modifications and variations to the present disclosure may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only and is not intended to limit the invention so further described in such appended claims.

1. An open time additive composition, comprising:
a first open time additive having the structure of Compound I, a salt thereof, or both; and
a second open time additive having the structure of Compound I, a salt thereof, or both;
wherein Compound I has the following formula: 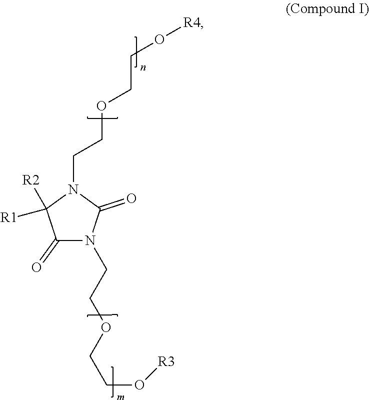
wherein, for both the first and second open time additives, m is an integer no less than zero, n is an integer no less than zero, and R1 and R2 are independently a branched or linear carbon chain having no less than one and no more than forty carbon atoms,
wherein for both the first and second open time additives, each carbon atom in R1 and R2 is independently substituted with one or more hydrogen atom, one or more hydroxyl group, one or more other carbon atom in the branched or linear carbon chain, an aryl group, or a combination thereof,
wherein, for both the first and second open time additives, each of R3 and R4 are independently a hydrogen atom or an ester group,
wherein, for the first open time additive, a sum of m and n is no greater than five, and
wherein, for the second open time additive, the sum of m and n is no less than fifteen.

2. The open time additive composition of claim 1, wherein m is an integer no less than five and no greater than one hundred for the second open time additive.

3. The open time additive composition of claim 1, wherein n is zero for the first open time additive.

4. The open time additive composition of claim 1, wherein, for the first open time additive:
n is no greater than five; and
m is no greater than five.

5. The open time additive composition of claim 1, wherein R3, R4, or both is the ester group for one or both of the first and second open time additives.

6. The open time additive composition of claim 1, wherein R1 and R2 are both methyl groups for one or both of the first and second open time additives.

7. The open time additive composition of claim 1, wherein the ester group is an oleate group having the following formula:

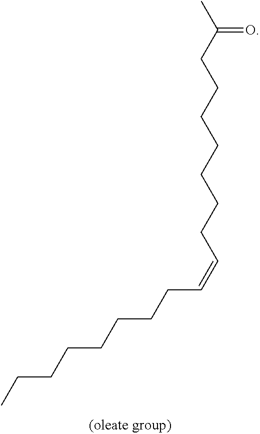 [SMILES: CCCCCCCC/C=C\CCCCCCCC(C)=O]

8. The open time additive composition of claim 1, wherein a weight ratio of the first open time additive to the second open time additive is from 1:9 to 9:1.

9. The open time additive composition of claim 1, wherein a weight ratio of the first open time additive to the second open time additive is from 1:3 to 3:1.

10. The open time additive composition of claim 1, wherein a weight ratio of the first open time additive to the second open time additive is from 1:2 to 2:1.

11. An architectural paint composition, comprising:
a solvent;
a latex binder; and
the open time additive composition of claim 1.

12. The architectural paint composition of claim 11, wherein the latex binder comprises an acrylate.

13. The architectural paint composition of claim 11, wherein the solvent is water.

14. The architectural paint composition of claim 11, wherein a concentration of the open time additive in the architectural paint composition is no less than about a tenth of a percent and no greater than about five percent based on the total weight of the architectural paint composition.

15. The architectural paint composition of claim 11, wherein the architectural paint composition has a solids content no less than about ten percent and no greater than about seventy percent based on the total weight of the architectural paint composition.

16. The architectural paint composition of claim 11, wherein the open time additive and the latex binder have a weight ratio of 1:999 to about 100:900 based on the total weight of the open time additive to the total weight of the latex binder.

17. The architectural paint composition of claim 11, wherein the architectural paint composition displays an open time increase of no less than ten percent compared to a baseline open time displayed for a baseline architectural paint, and
wherein the baseline architectural paint does not include the open time additive and has a relative composition for other components included in the architectural paint composition that is approximately the same, and wherein the open time is determined according to OTA test ASTM D7488-11 “Standard Test Method for Open Time of Latex Paints”.

18. The architectural paint composition of claim 11, wherein the architectural paint composition has a volatile organic compound, VOC, content of less than one thousandth of a percent based on the total weight of the architectural paint composition, and wherein VOC content is determined according to EPA Method 24.
